(19) United States
(12) Patent Application Publication
(10) Pub. No.: US 2008/0177278 A1
(43) Pub. Date: Jul. 24, 2008

(54) MEDICAL DEVICE FOR DETERMINING THE POSITION OF INTRACORPOREAL IMPLANTS

(75) Inventors: Roman Stauch, Assamstadt (DE); Miroslaw Wrobel, Karlstadt (DE)

Correspondence Address: BACHMAN & LAPOINTE, P.C., 900 CHAPEL STREET, SUITE 1201, NEW HAVEN, CT, 06510, US

(73) Assignee: WITTENSTEIN AG, Igersheim (DE)

(21) Appl. No.: 11/952,253

(22) Filed: Dec. 7, 2007

(30) Foreign Application Priority Data
Dec. 13, 2006 (DE) 10 2006 059 226.3

(51) Int. Cl. A61B 19/00

(52) U.S. Cl. 606/129

(57) ABSTRACT

Magnetically locatable implants, such as fixation systems, anchor plates or the like, and the determination of their intracorporeal position by means of a medical device comprising at least one impedance-measuring device and a device for generating and coupling a magnetic field which are both connected to at least one intracorporeal electrically conductive probe, the impedance-measuring device being additionally equipped with at least one connector for connection to the magnetically locatable implant. The device is particularly suitable for determining the position of magnetically locatable pedicle screws and the like in a spinal column fixation system with rod-shaped electrically conductive probes as stabilizers.

### BACKGROUND OF THE INVENTION

The invention relates, on the one hand, to a surgical implant made of plastic, ceramic or the like and, on the other hand, to a medical device for determining its intracorporeal position.

An implant is a synthetic material implanted into the body in human or veterinary medicine and intended to remain there either permanently or at least over a long period of time. The medical implants are intended to support or replace the functions of the body.

By virtue of the latest technology, there are now also surgical implants made of plastic, ceramic, carbon fibers, etc., such that these materials too are now often used in so-called fixation systems, medullary nails, plates and the like, where screws with an eyelet, referred to as pedicle screws, are used which can be made, for example, mainly of plastic and through whose respective eyelets the fixation rods are inserted. These fixation systems are used, for example, in spinal fractures, which may be caused by falls, osteoporosis, tumors, etc.

In the conventional operating procedure, the pedicle screws and fixation rods are introduced by detaching large areas of the muscles from the spinal column, the pedicle screws being screwed into the vertebral body of the spinal column on both sides of the vertebral canal, and the fixation rods being inserted from the top downward through the eyelets of the pedicle screws.

In young patients, the fixation rods are removed after about six months, since otherwise there is a danger of the screws/rods breaking with the increasing mobility of the patient.

In older patients, for example with osteoporotic damage (fractures, sintering) of the spinal column, permanent fusion is often generally carried out by treatment with a rod system (internal fixator, optionally in combination with bone cement) or a plate system according to the fixator principle. However, there is a danger here of the screws breaking through into the intervertebral disc space because of the reduced bone strength in osteoporosis.

By contrast, in the so-called minimally invasive method, the spinal column is not exposed, and instead the treatment is performed from the outside, only through small incisions in the skin. This method includes, for example, vertebroplasty, in which bone cement is injected into the vertebral bodies without these being straightened beforehand, or kyphoplasty, in which collapsed vertebrae are straightened with the aid of an inflatable balloon and then with injected biological cement. By contrast, the introduction of fixation rods in the treatment of fractures of the vertebral bodies has not hitherto been possible by a minimally invasive procedure.

The object of the invention is to make it possible, in a simple and reliable way, to determine the position of guide bores or locking bores for implants made of plastic, ceramic or the like, without the need for major surgical procedures.

### SUMMARY OF THE INVENTION

According to the invention, this object is firstly achieved by the fact that the surgical implants of plastic, ceramic or the like are designed to be magnetically locatable, and, secondly, by using a medical device comprising at least one impedance-measuring device and a device for generating and coupling a magnetic field which are both connected to at least one intracorporeal probe, the impedance-measuring device additionally being equipped at least with one connector for electrically conductive connection to the magnetic part of the implant.

According to the invention, the measurement parameter used in the impedance measurement for determining the position is the phase displacement between current and voltage that occurs as a function of the distance between the intracorporeal metal probe and the magnetically locatable part of the implant, i.e. if it is small, this means a small distance, and if it is large, this means a large distance. As a result, the size of the impedance is a function of the distance between probe and implant.

The combination of impedance measurement and magnetic field measurement has the advantage that the impedance-measuring device can be used for a rough determination of position and the device for generating and coupling a magnetic field can be used for a precise determination of position, where a rough determination of position is to be understood as the first approach of the probe to the implant, whereas the remaining path of the probe as far as the implant, or even into the implant, is effected by precise determination of position with the aid of the device for generating and coupling a magnetic field.

In other words, taking the analogy of the lighting of a vehicle: “full beam” for the rough positioning by approach via the impedance measurement, where the tissue composition can also be measured, i.e. which tissue is passed and what vitality it has. As soon as a relative approach to the implant is achieved, a switch is made to “dimmed beam” or to the magnetic field for the more precise positioning.

If several implants are present one behind another for example, it is expedient for the impedance-measuring device and the device for the magnetic field to be switched on alternately, i.e. the rough determination of position and the precise determination of position take place anew for each consecutive implant.

It is also possible for the impedance-measuring device and the device for the magnetic field to be switched in such a way that they are simultaneously active, which improves the guiding of the probe.

The main advantage of the medical device according to the invention lies in the use in minimally invasive surgery, since extensive areas of tissue and/or muscle no longer have to be detached, thus resulting in reduced blood loss, less damage to the muscle nerves, preservation of the proprioreception, less scar formation on the skin and muscles, and therefore shorter periods of confinement and earlier mobilization of the patients. Finally, the X-ray burden for physician and patient during intraoperative application can also be reduced by the use of magnetic field technology.

The device according to the invention is suitable in particular in minimally invasive surgery in a spinal column fixation system with metal rods as stabilizers and with so-called magnetically locatable pedicle screws, the rods serving specifically as probes with intracorporeal capability with which the impedance-measuring device and the device for the magnetic field are connected, while the additional connector of the impedance-measuring device is electrically conductively connected to the magnetically locatable pedicle screws.

It is expedient for the intracorporeal probe to have a rod-shaped design and, at its free hand end, to have an element for generating a spatial, possibly constant magnetic field (solenoid), this being secured to the probe by a thread, a quick coupling or the like, such that this can be removed again at the end of an operation. At the same time, however, it must be ensured that this threaded connection cannot come loose during manipulation in an operation, which in particular must not be allowed to happen upon rotation of the probe to the left and right, for which reason a suitable fine thread is to be preferred here.

The device according to the invention is operated as follows: first, during its intracorporeal introduction, the probe is used for a rough determination of position, specifically with the aid of the impedance-measuring device measuring the phase displacement between current and voltage as a function of the distance between the probe and the implant, whereas, shortly before the implant is reached by the probe, a precise determination of position is carried out in a known manner with the aid of the device for the magnetic field.

### BRIEF DESCRIPTION OF THE DRAWINGS

Further features and advantages of the present invention will become clear from the following description of an illustrative embodiment in which reference is made to the drawing, in which:

FIG. 1 shows a side view of a schematically illustrated portion of a spinal column, where three vertebral bodies can be connected to one another in accordance with the invention, i.e. can be stabilized and

FIG. 2 shows a schematic plan view of a so-called pedicle screw made of plastic, with an inserted metal bushing, as is used in the example in FIG. 1 for stabilizing the vertebral bodies.

### DETAILED DESCRIPTION

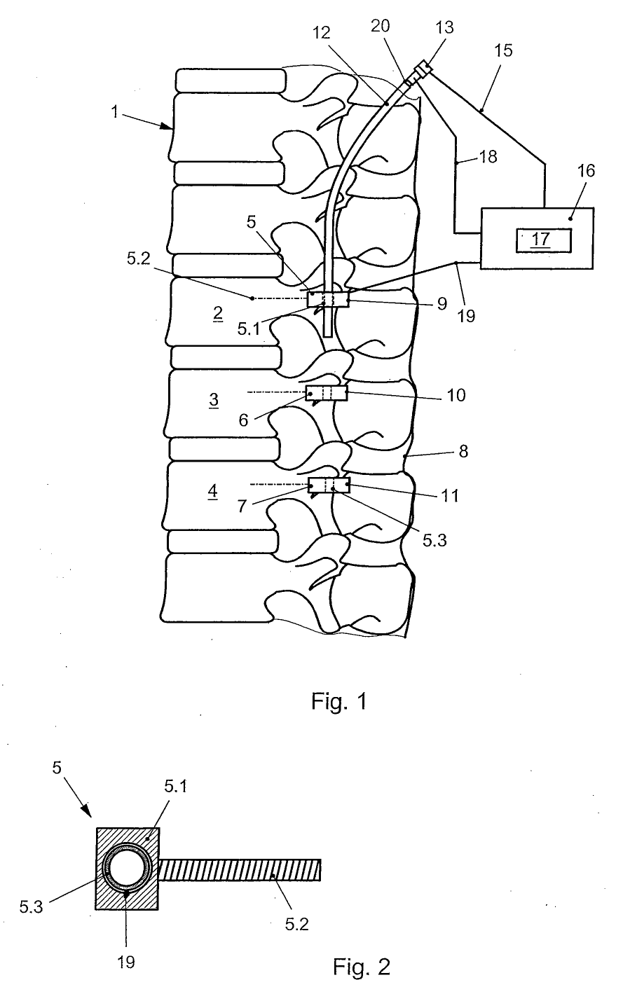

In detail, FIG. 1 shows a lateral portion of a spinal column 1 in three vertebral bodies 2-4 of which so-called pedicle screws 5-7 are screwed, for which purpose the skin designated by 8 has a relatively small incision, indicated by 9-11. The individual pedicle screws 5-7 are composed mainly of plastic and, as seen from the example of the first screw 5, an eyelet 5.1, a threaded shaft 5.2 and a metal bushing 5.3 (FIG. 2) which is inserted into the eyelet 5.1 and is made of titanium, it also being possible to use chromium and cobalt/nickel alloys.

Besides the example in FIG. 2, the present invention also extends to other surgical implants such as medullary nails, anchor plates, screws, implants that have to be connected to a bone, etc. All these implants, which can be produced from plastic, ceramic or the like, have one thing in common, namely they have to be designed to be magnetically locatable, i.e. they must have at least one electrically conductive marker, such as a metal insert, metal fibers, metal powder, etc.

The three vertebral bodies 2-4 according to FIG. 1 are intended to be connected to one another, i.e. stabilized, for which purpose, according to the minimally invasive method, an electrically conductive fixation rod 12 is used which at its head end has a solenoid 13, which is connected via an electrical lead 15 to an impedance-measuring device 16 with an optical and/or acoustic display 17. At the same time, the impedance-measuring device 16 is connected via the lead 18 to the fixation rod 12 and via a connector 19 to the sleeve 5.3 of the pedicle screw 5.

The minimally invasive placement of the pedicle screws 5-7 and of the fixation rod 12 takes place as follows. In FIG. 1 only a half of the screw arrangement is shown, since in practice a screw is applied on both sides of the vertebral canal of the vertebral bodies 2-4 concerned, with the result that two rows of screws are present, each with a fixation rod 12, of which, however, only one row can be seen in FIG. 1.

To insert the pedicle screws 5-7, suitably short incisions 9-11 are made in the skin 8, and the pedicle screws 5-7 are screwed, after preliminary drilling, with their respective threaded shaft (example: 5-5.2) into the vertebral bodies 2-4 (example: 2) in such a way that their eyelets (example: 5.1) are horizontal. It should be noted here that the screws 5-7 are placed through the respective muscle, i.e for this purpose the muscle should if possible be divided longitudinally or pushed aside.

For minimally invasive introduction of the fixation rod 12 through the metal sleeves of the pedicle screws 5-7 which are otherwise made of plastic, the rod should also if possible be pushed through the muscle. At the start, the skin 8 must be penetrated by an incision (not shown).

To start with, a targeting aid for the fixation rod 12 lies in the impedance measurement in which the measurement parameter used is the phase displacement between current and voltage that can be read off via the display 17 and/or acoustically perceived, i.e the smaller this phase displacement, the closer the tip of the fixation rod 12 is to the metal sleeve 5.3 of the eyelet 5.2. When this “A small” has reached a defined near area, the device is switched to the magnetic field, i.e. to the solenoid for example (current flow through bar magnet), since with the latter the fixation rod 12 can be guided with greater precision and thus more easily through the sleeve 5.3.

The continued guiding of the fixation rod 12 through the sleeves of the two further pedicle screws 6 and 7 takes place analogously, and the lead 19 then has to be connected to the corresponding sleeves of each of these screws. Two possibilities can be selected here, specifically, in the first case, the impedance method as rough orientation and then, for more precise approach and guiding through the eyelets, the device for generating and coupling the magnetic field or both.

The solenoid 13 is secured on the fixation rod 12 as probe by means of a fine thread, a quick coupling or the like, specifically such that this unit can be removed again at the end of the intervention.

Moreover, the remaining portion of the fixation rod 12 in the spinal column 1 has a notch 20 on its circumference, such that this portion can be removed again in due course with the aid of a suitable extraction instrument.

Overall, therefore, the present invention represents a further milestone in minimally invasive surgery to the benefit of patients.

1. Surgical implants made of plastic and/or ceramic wherein the implants include means for magnetically locating the implants.

2. Surgical implant according to claim 1, wherein the implants comprise at least one electrically conductive marker selected from the group consisting of metal inserts, metal fibers, carbon fibers, metal powder, and mixtures thereof.

3. Medical device for determining the position of intracorporeal implants comprises at least one impedance-measuring device (16) and a device for generating and coupling a magnetic field (13) which are both connected to at least one intracorporeal probe (12), the impedance-measuring device (16) being additionally equipped with at least one connector (19) for connection to the conductive part of the implant (5).

4. Medical device according to claim 3, wherein the impedance-measuring device comprises means for determining the position of an implant (5) by measuring the phase displacement between current and voltage which occurs as a function of distance between the intracorporeal probe (12) and the magnetic part of the implant (5).

5. Medical device according to claims 4, wherein the impedance-measuring device (16) is used for a rough determination of position and the device for generating and coupling a magnetic field (13) is used for a precise determination of position.

6. Medical device according to claim 5, including means for switching the impedance-measuring device (16) and the device for generating and coupling a magnetic field (13) on alternately.

7. Medical device according to claim 5, including means for switching the impedance-measuring device (16) and the device for generating and coupling a magnetic field (13) in such a way that they are simultaneously active.

8. Medical device according to claim 3, wherein the intracorporeal probe (12) has a rod-shaped design and at a free hand end, has an element for generating a magnetic field.

9. Medical device according to claim 8, wherein the element comprises a solenoid (13) secured to the probe (12) by a thread.

10. Medical device according to claim 8, wherein the intercorporeal part of the rod-shaped probe (12) has, at least at one end, a depression (20) for an attachment of an extraction instrument.

11. Medical device for determining the position of magnetically locatable pedicle screws in a spinal column fixation system with rod-shaped probes as stabilizers comprises an intracorporeal electrically conductive probe (12) connected to an impedance-measuring device (16) and to a device for generating and coupling a magnetic field (13), and an additional connector (19) of the impedance-measuring device (16) is electrically conductively connected to respective magnetically locatable pedicle screws.

12. Method for determining the position of intracorporeal magnetically locatable implants with the aid of a device comprising at least one impedance-measuring device (16) and a device for generating and coupling a magnetic field (13) which are both connected to at least one intracorporeal probe (12), the impedance-measuring device (16) additionally being equipped with at least one connector (19) for connection to the magnetically locatable implant (5), comprising the steps of first using the probe (12), upon introduction into the body, to carry out a rough determination of position by means of the impedance-measuring device (16) measuring the phase displacement between current and voltage as a function of the distance between the probe (12) and the magnetically locatable implant (5), whereas, shortly before the magnetically locatable implant (5) is reached by the probe (12), a precise determination of position takes place with the aid of the device for generating and coupling a magnetic field (13).

13. Method according to claim 12, wherein the rough determination of position and thereafter the precise determination of position are carried out successively on several consecutively arranged implants.

14. Method according to claim 12, wherein the determination of position in one or more consecutively arranged is carried out simultaneously by the impedance-measuring device (16) and the device for generating and coupling a magnetic field (13).
